(19) United States
(12) Patent Application Publication
(10) Pub. No.: US 2002/0071474 A1
(43) Pub. Date: Jun. 13, 2002

(54) Device for measuring temperature of vessel walls

(76) Inventor: Randell L. Werneth, Powey, CA (US)

Correspondence Address: ROBROY R FAWCETT, 1576 KATELLA WAY, ESCONDIDO, CA, 92027, US

(21) Appl. No.: 09/939,210

(22) Filed: Aug. 24, 2001

Related U.S. Application Data
(60) Provisional application No. 60/246,951, filed on Nov. 10, 2000.
(60) Provisional application No. 60/268,341, filed on Feb. 12, 2001.
(60) Provisional application No. 60/246,951, filed on Nov. 10, 2000.
(60) Provisional application No. 60/268,341, filed on Feb. 12, 2001.

(51) Int. Cl. G01K 13/00; G01K 7/04; A61B 5/00

(52) U.S. Cl. 374/179; 374/166; 374/148; 600/549

(57) ABSTRACT

The present invention may be embodied in a device for detection of vulnerable plaque in a vessel. Vulnerable plaque is detected in the vessel based on a temperature increase of the vessel's wall.

### CROSS REFERENCE TO RELATED APPLICATIONS

This application claims priority under 35 U.S.C. § 119(e)(1) and 37 C.F.R. 1.78(a)(4) to U.S. provisional patent application serial No. 60/246,951 filed Nov. 10, 2000, and to U.S. provisional patent application serial No. 60/268,341 filed Feb. 12, 2001. The entire disclosures of U.S. provisional patent applications serial Nos. 60/246,951 and 60/268,341 are incorporated herein by reference.

### BACKGROUND OF THE INVENTION

1. Field of the Invention

The present invention relates to diagnosis and treatment of vulnerable plaque in blood vessels.

2. Description of the Prior Art

Vulnerable plaque rupture is believed to be the cause of death in a large percentage of patients suffering heart attack and stroke. Detection of vulnerable plaque is problematic because the vulnerable plaque may not be associated with arterial blockage or the like. Currently, arterial blockage is detected using radiographic techniques.

Accordingly, there exists a need for an apparatus and related techniques for diagnosing and treating vulnerable plaque in blood vessels. The present invention satisfies these needs and provides further related advantages.

### SUMMARY OF THE INVENTION

The present invention may be embodied in device for detecting vulnerable plaque in a vessel by measuring the temperature of the vessel's inner wall. The device may be a catheter having a delivery tube, an inner wire assembly, and a plurality of sensing elements. The delivery tube has a distal end with an opening. The plurality of sensing elements is coupled to the inner wire assembly. The inner wire assembly is placed within the delivery tube and is extendable through the opening at the distal end of the delivery sheath to release the sensing elements to contact the vessel's inner wall for sensing the temperature of the vessel's inner wall.

In more detailed features of the inventions, the sensing elements may be thermo-sensing contact junctions. Also, the plurality of sensing elements may comprise at least eight sensing elements.

### BRIEF DESCRIPTION OF THE DRAWINGS

The accompanying drawings illustrate embodiments of the present invention and, together with the description, serve to explain the principles of the invention.

FIG. 1 shows a cross-sectional elevation view of a device for detecting vulnerable plaque within a vessel, according to the present invention.

FIG. 2 shows a second cross-sectional elevation view of the device for detecting vulnerable plaque of FIG. 1, according to the present invention.

### DETAILED DESCRIPTION

The present invention may be embodied in a device 10 for detecting vulnerable plaque in vessels. Vulnerable plaque is defined as a lipid-rich atheromatous core, covered by a fibrous cap, and the presence of on-going inflammation within and underneath the cap. As inflammation reactions occur in the atheromatous core and in the thin-film fibrotic cap, a local increase in temperature in the vessel may occur.

Thermography may provide a technique to detect the vulnerable plaque. Application of cryoenergy to the vulnerable plaque may provide a technique to deactivate the inflammatory response and thus stabilize the associated lesion. The application of cryoenergy to the vulnerable plaque creates an injury in the vessel that destroys the cellular messenger apparatus of the mitochondria while preserving the anhydrous structure of the cellular matrix. Cryoenergy may eradicate components of the vulnerable plaque and allow a natural healing by migration of normal smooth muscle cells from regions adjacent to the treatment site.

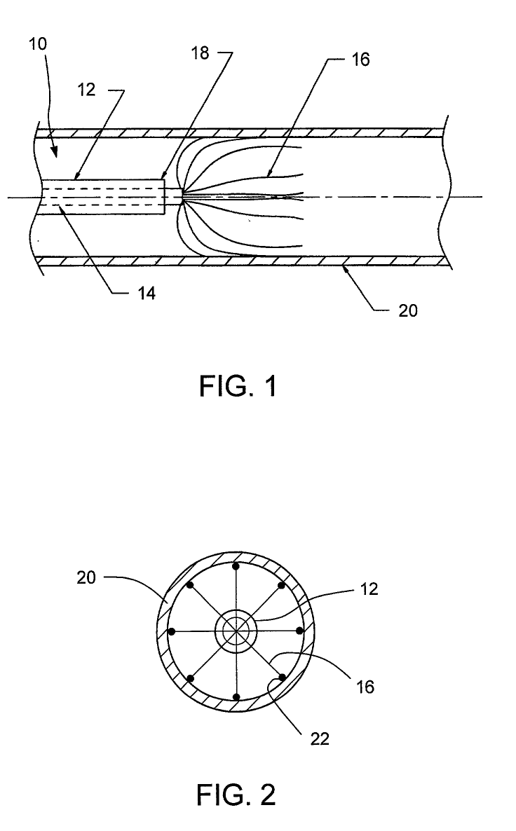

With reference to FIGS. 1 and 2, a device for detecting of vulnerable plaque of the present invention may be embodied in a catheter 10 having a delivery sheath 12 or tube, an inner wire assembly 14, and a plurality of sensing elements 16. Initially, the sensing elements and inner wire are contained within the delivery sheath. A distal tip 18 or end of the delivery sheath may be guided within a vessel 20 until the catheter is located at a place of concern for vulnerable plaque. A radiopaque tip-marker may be placed at the distal tip or end of the delivery sheath.

To make a temperature measurement of the vessel's inner wall, the delivery sheath 12 is pulled back with respect to the inner wire 14. The sensing elements 16 are coupled to the end of the inner wire and are released as the inner sheath is pulled back. Once released, the sensor elements contact the vessel's inner walls. The sensor elements gently contact the vessel's inner wall allowing temperature measurement without causing cellular rupture of the vessel wall.

The sensing elements 16 may be temperature sensing wire sensors, fiber sensors and thermo-sensing contact junctions 22. Advantageously, at least eight (8) sensing elements are used. The inner wire assembly 14 connects each sensing element to monitoring equipment (not shown). Each sensing element sends a signal to the monitoring equipment indicating the wall temperature at the sensor element's location.

Once a higher (or lower than) normal temperature is detected, indicative vulnerable or hot plaque, the location of the catheter 10 is secured and the inner wire 14 and sensing elements 16 are removed from the delivery sheath 12. The delivery sheath is left in place within the vessel 20 as a place marker for the location of the sensed temperature difference. A treatment catheter may be placed at the sensed location using the delivery sheath as a guide-wire device.

The catheter 10 of the invention may be measure temperature differences within vessels °such as arteries or veins of the vascular system.

While the invention has been illustrated and described in detail in the drawings and foregoing description, it should be understood the invention may be implemented though alternative embodiments within the spirit of the invention. Thus, the scope of the invention is not intended to be limited to the illustration and description in this specification, but is to be defined by the appended claims.

We claim:

1. A catheter for sensing inner-wall temperature of a vessel, comprising:
a delivery tube having a distal end with an opening;
an inner wire assembly for placement within the delivery tube; and
a plurality of sensing elements coupled to the inner wire assembly;
wherein the inner wire assembly is extendable through the opening at the distal end of the delivery sheath to release the sensing elements to contact the vessel's inner wall for sensing the temperature of the vessel's inner wall.

2. A catheter for sensing inner-wall temperature of a vessel as defined in claim 1, wherein the sensing elements are thermo-sensing contact junctions.

3. A catheter for sensing inner wall-temperature of a vessel as defined in claim 1, wherein the plurality of sensing elements comprise at least eight sensing elements.
